(19) United States
(12) Patent Application Publication
(10) Pub. No.: US 2012/0041261 A1
(43) Pub. Date: Feb. 16, 2012

(54) BALLOON CATHETER AND BALLOON CATHETER ASSEMBLY

(75) Inventors: Iwayoshi SHUNSUKE, Kanagawa (JP); Sekine YUSUKE, Kanagawa (JP)

(73) Assignee: Terumo Kabushiki Kaisha, Shibuya-ku (JP)

(21) Appl. No.: 13/237,372

(22) Filed: Sep. 20, 2011

Related U.S. Application Data
(63) Continuation of application No. PCT/JP2010/053826, filed on Mar. 9, 2010.

(30) Foreign Application Priority Data
Mar. 25, 2009 (JP) 2009-075218

(51) Int. Cl. A61B 1/00

(52) U.S. Cl. 600/115

(57) ABSTRACT

A balloon catheter includes: an outer catheter; an inner catheter inserted in the outer catheter, capable of moving relative to the outer catheter in the longitudinal direction thereof, and permitting a flexible medical elongate member to be inserted therethrough; an inflatable and contractible balloon constructed from a film member, having one end portion and the other end portion fixed respectively to a distal portion of the outer catheter and a distal portion of the inner catheter, and projecting from a distal end of the outer catheter by an amount which varies according to the amount of movement of the inner catheter relative to the outer catheter; and a position restrictor for restricting the position of a distal end of the medical elongate member, which is inserted through the inner catheter, relative to a distal end of the balloon.

This application is a continuation of International Application No. PCT/JP2010/053826 filed on Mar. 9, 2010, and claims priority to Japanese Application No. 2009-075218 filed on Mar. 25, 2009, the entire content of both of which is incorporated herein by reference.

### TECHNICAL FIELD

The present invention generally relates to a balloon catheter and a balloon catheter assembly provided with such balloon catheter.

### BACKGROUND DISCUSSION

Conventionally, in falloposcopic tuboplasty for treating obstruction or stenosis of a fallopian tube under falloposcopy, a balloon catheter is used with a falloposcope. A known balloon catheter used in this regard includes an outer catheter, an inner catheter inserted in the outer catheter and movable along the longitudinal direction thereof, and an inflatable and contractible balloon member for connecting a distal portion of the inner catheter and a distal portion of the outer catheter to each other. An example of this catheter is disclosed in Japanese Patent No. 2813463.

The balloon member in the balloon catheter described in this patent projects from the distal end of the outer catheter by an amount which varies according to the amount of movement of the inner catheter relative to the outer catheter, and the balloon member is inflated and contracted in the projecting state. The balloon catheter configured in this way is used in the state in which a falloposcope is inserted through the inner catheter.

The disclosed balloon catheter also includes graduations indicative of the position of the falloposcope relative to the balloon member in the longitudinal direction of the catheter. Even with the graduations thus provided, however, misreading of the graduations can occur, and so the falloposcope may excessively project from the distal end of the balloon member, possibly resulting in damage of the fallopian tube by the projecting falloposcope or damage (cracking) of the falloposcope itself in contact with the fallopian tube.

### SUMMARY

According to one aspect, a balloon catheter includes an outer catheter; an inner catheter positioned in the outer catheter, movable relative to the outer catheter in the longitudinal direction, and permitting a flexible medical elongate member to be inserted therethrough; an inflatable and contractible balloon constructed from a film member, and having one end portion and the other end portion fixed respectively to a distal portion of the outer catheter and a distal portion of the inner catheter, and projecting from a distal end of the outer catheter by an amount which varies according to the amount of movement of the inner catheter relative to the outer catheter; and position restriction means for restricting the position of a distal end of the medical elongate member, which is inserted through the inner catheter, relative to a distal end of the balloon. The construction of the balloon catheter helps ensure that a medical elongate member inserted through the inner catheter of the balloon catheter is inhibited or prevented from projecting in excess beyond the distal end of the balloon. Consequently, at a limit of movement of the medical elongate member in the distal direction, for example, the distal end of the medical elongate member is prevented from projecting in excess from the distal end of the balloon.

The position restriction means restricts the position of the distal end of the medical elongate member so that, at a limit of movement of the medical elongate member in the distal direction, the distal end of the medical elongate member is located in the vicinity of the distal end of the balloon. This helps ensure that the distal end of the medical elongate member, which is inserted through the inner catheter of a balloon catheter assembly, is reliably inhibited or prevented from projecting in excess from the distal end of the balloon.

The position restriction means can include: a contact member disposed on the proximal side of the inner catheter, and having a contact surface with which a part of the medical elongate member can make contact; and a connection mechanism by which the contact member and the inner catheter are connected to each other so that they can be brought close to (can move toward) and away from each other.

The connection mechanism can be configured to connect the contact member and the inner catheter to each other in such a manner that when the inner catheter and the contact member are moved in the same direction, the amount of movement of the contact member is less than (e.g., one-half) the amount of movement of the inner catheter.

The connection mechanism can include: at least a pair of pulleys arranged on the contact member along the longitudinal direction of the inner catheter; and a linear member or belt-like member which is wrapped around the pair of pulleys and to which the inner catheter and the outer catheter are fixed. Alternatively, the connection mechanism can include: at least one pinion gear rotatably supported on the contact member; an inside rack provided on the inner catheter and engaging with the pinion gear; and an outside rack provided on the outer catheter so as to face the inside rack, with the pinion gear therebetween, and engaging with the pinion gear.

The contact member can be constructed so that the position of the contact surface can be finely adjusted along the longitudinal direction of the catheter. This helps ensure that the position of the distal end of the medical elongate member relative to the distal end of the balloon is adjustable, for example in such a manner that the distal end of the medical elongate member is located a little on the proximal side relative to the distal end of the balloon, or so that the distal end of the medical elongate member is located a little on the distal side relative to the distal end of the balloon.

The position restriction means can also include a lock member for maintaining a state in which the contact member and the part of the medical elongate member, capable of making contact with the contact member, are in contact with each other. This helps reliably maintain the state in which the contact member and the medical elongate member are in contact with each other. Consequently, it is possible to relatively assuredly maintain a state in which, for example, the distal end of the balloon and the distal end of the medical elongate member are located at the same position with respect to the longitudinal direction of the catheter.

The balloon catheter can additionally be provided with projection preventive means for preventing the distal end of the inner catheter from projecting beyond the distal end of the outer catheter. This makes it possible to fairly securely prevent the distal end of the inner catheter from inadvertently projecting from the distal end of the outer catheter.

According to another aspect, a balloon catheter assembly comprises: a longitudinally extending outer catheter possessing a distal portion; an inner catheter positioned in the outer catheter and movable relative to the outer catheter in a longitudinal direction of the outer catheter, with the inner catheter possessing a distal portion; an inflatable and contractible balloon constructed from a film member having one end portion fixed to the distal portion of the outer catheter and an other end portion fixed to a distal portion of the inner catheter, with the balloon projecting distally beyond a distal-most end of the outer catheter by an amount which varies according to an amount of relative longitudinal movement between the inner catheter and the outer catheter; an elongated medical member positioned in the inner catheter of the balloon catheter and being movable relative to the inner catheter, the elongated medical member possessing a distal-most end; and position restriction means for restricting a position of the distal-most end of the medical elongate member positioned in the inner catheter relative to the distal end of the balloon.

According to a further aspect, a balloon catheter comprises: a longitudinally extending outer catheter possessing a distal portion; an inner catheter positioned in the outer catheter and axially movable relative to the outer catheter in a distal direction and proximal direction relative to the outer catheter, the inner catheter having a through hole configured to receive an elongated medical member, the inner catheter possessing a distal portion; an inflatable and contractible balloon constructed from a film member and projecting distally beyond a distal-most end of the outer catheter, the film possessing one end portion fixed to the distal portion of the outer catheter and an other end portion fixed to the distal portion of the inner catheter so that axial movement of the inner catheter in the distal direction relative to the outer catheter from a first position to a second position spaced apart from each other by a first axial distance causes the balloon to project further distally beyond the distal-most end of the outer catheter, an axial distance between a distal-most end of the balloon at the first position and the distal-most end of the balloon at the second position being a second distance less than the first distance; a contact portion configured to be contacted by a part of the elongated medical member when the elongated medical member is moved relative to the contact portion; and a connector connecting the contact portion and the inner catheter so that the axial movement of the inner catheter relative to the outer catheter over the first axial distance causes movement of the contact portion over an axial distance no greater than the second axial distance.

### BRIEF DESCRIPTION OF DRAWINGS

FIGS. 1(a) and 1(b) are partial longitudinal cross-sectional views of a first embodiment of a balloon catheter assembly (including a balloon catheter) disclosed here, and sequentially illustrating states of use of the assembly including the balloon catheter.

FIG. 2 is a partial longitudinal cross-sectional view of the first embodiment of the balloon catheter assembly disclosed here illustrating a further state of use.

FIG. 3 is a longitudinal cross-sectional view showing a limit of movement in the distal direction of an inner catheter in the balloon catheter assembly shown in FIG. 1.

FIGS. 4(a) and 4(b) are partial longitudinal cross-sectional views illustrating a second embodiment of a balloon catheter assembly (including a balloon catheter) disclosed here.

FIG. 5 is a partial longitudinal cross-sectional view illustrating a third embodiment of a balloon catheter assembly (including a balloon catheter) disclosed here.

FIGS. 6(a) and 6(b) are partial longitudinal cross-sectional views illustrating a fourth embodiment of a balloon catheter assembly (including a balloon catheter) disclosed here.

FIG. 7 is a longitudinal sectional view illustrating a part of a balloon catheter assembly according to a fifth embodiment disclosed here.

FIG. 8 is a longitudinal sectional view illustrating a part of a balloon catheter assembly according to a sixth embodiment disclosed here.

FIG. 9 is a longitudinal sectional view illustrating a part of a balloon catheter assembly according to a seventh embodiment disclosed here.

FIGS. 10(a)-10(c) are partial longitudinal cross-sectional views illustrating steps in use of the balloon catheter assembly shown in FIG. 1.

### DETAILED DESCRIPTION

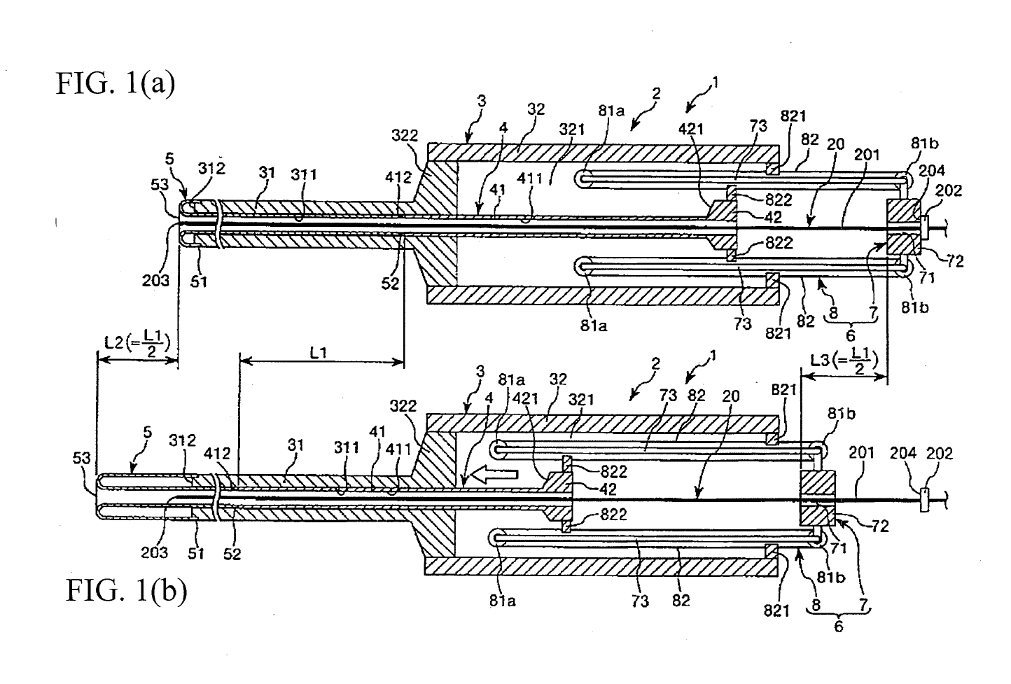

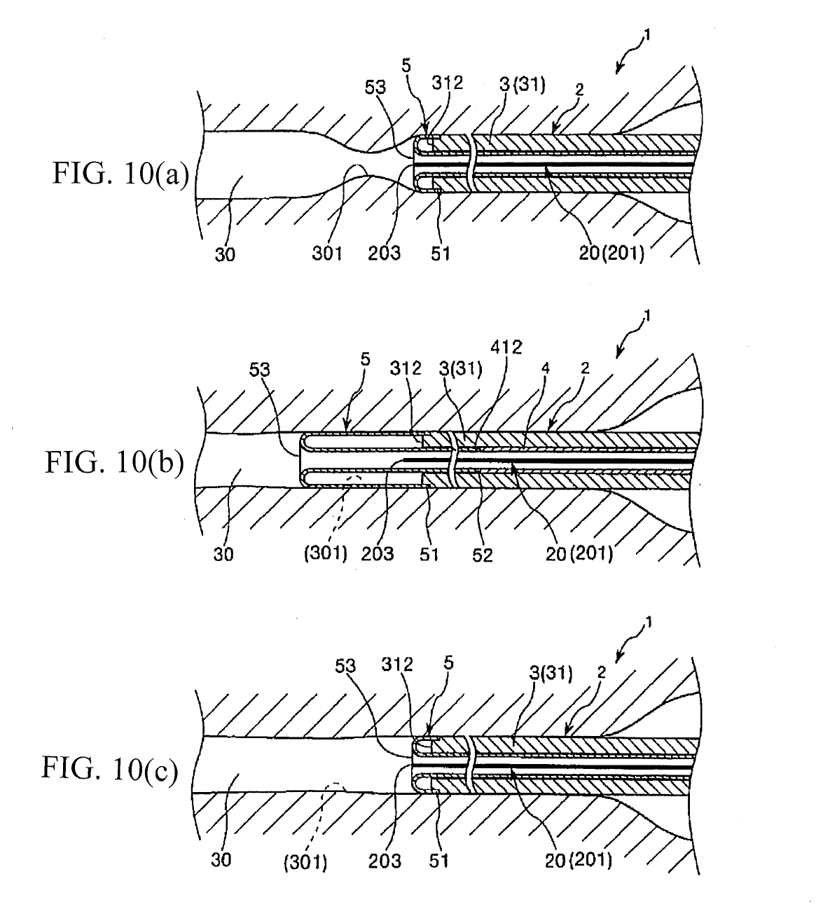

A first embodiment of a balloon catheter assembly, including a balloon catheter, disclosed here is illustrated in FIGS. 1-3 which illustrate features and operational states of the assembly, and FIG. 10 which depicts steps associated with use of the balloon catheter assembly.

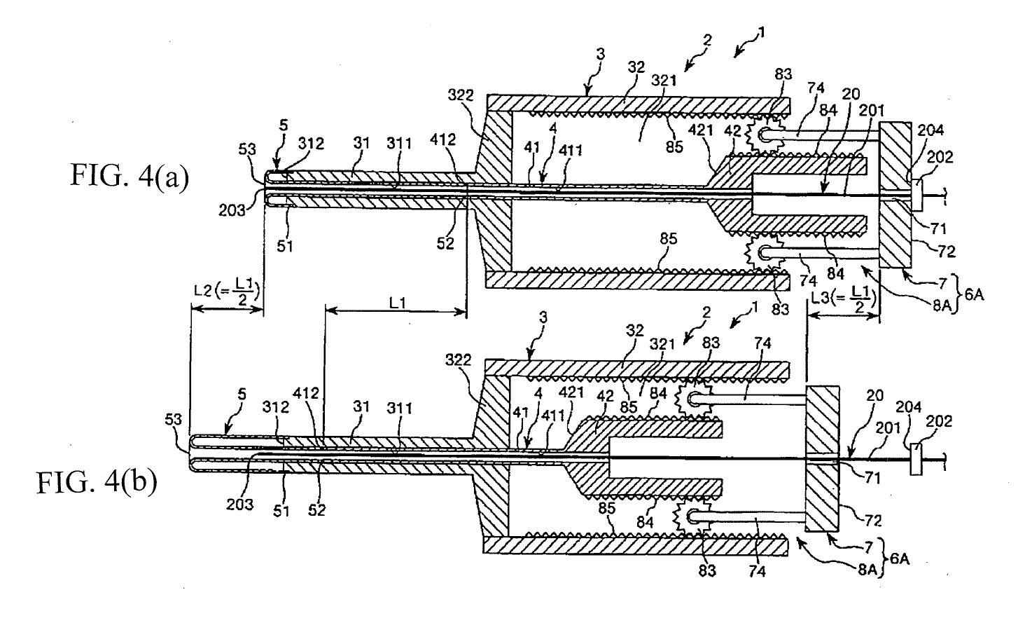

For convenience of description, the right side in FIGS. 1-3 and 10 (and in FIGS. 4-9 as well) is referred to as the “proximal (side)” and the left side is referred to as the “distal (side).”

The balloon catheter assembly 1 shown in FIGS. 1-3 is a balloon catheter assembly having particularly useful application in falloposcopic tuboplasty. The balloon catheter assembly 1 includes a balloon catheter 2, and a medical elongate member 20 in the form of a falloposcope 20. The falloposcope 20 is configured to be inserted through the balloon catheter 2. Set forth below is a description of the configuration and construction of each of those components.

The falloposcope 20 includes a falloposcope body 201 which is elongate and flexible, and an enlarged-diameter section or enlarged member 202 which is positioned at an intermediate part of the falloposcope body 201. The falloposcope body 201 has an image pick-up means having, for example, a CCD camera for picking up a pixel image, located on the distal side of the image pick-up means. The enlarged-diameter section 202 possesses an enlarged outer diameter which is larger in outer diameter than the adjoining portions of the falloposcope body 201.

The balloon catheter 2 includes an outer catheter 3, an inner catheter 4 positioned in the outer catheter 3, and a balloon 5 connecting the distal ends of the outer catheter 3 and the inner catheter 4 to each other.

The outer catheter 3 has an outer catheter body 31 which is elongate and flexible, and an outer hub 32 which is fixed to a proximal portion of the outer catheter body 31.

The outer catheter body 31 has a lumen (first lumen) 311 extending along the longitudinal direction (axial extent) of the outer catheter body 31. The inner catheter body 41 of the inner catheter 4 is inserted into or positioned in the lumen 311. The inner peripheral surface defining or surrounding the lumen 311 of the outer catheter body 31 may be a treated surface, for example a surface which has undergone a friction-reducing treatment, such as coating with a fluoro-polymer, to reduce frictional resistance between the inner peripheral surface and the outer peripheral surface of the inner catheter body 41 or the balloon 5.

In addition, the outer catheter 3 is connected to a fluid supply source which supplies a working fluid for inflation and contraction of the balloon 5. The working fluid from the supply source is supplied through the lumen 311 into the balloon 5. In addition to the lumen 311, a second lumen for supplying the working fluid may be formed in the outer catheter body 31 in parallel to the lumen 311.

The outer hub 32 is fixed to the proximal portion of the outer catheter body 31. The fixation method not specifically restricted. Possible fixation methods include, for example, a fusing method such as heat fusing (welding), high-frequency fusing, and ultrasonic fusing, an adhesion method such as adhesion with an adhesive or solvent, or the like. The outer catheter body 31 and the outer hub 32 may be integrally molded by, for example, injection molding or the like. The outer hub 32 is composed of a rectangular parallelepiped which is tetragonal in cross-section, and an inner cavity 321 of the outer hub 32 communicates with the lumen 311 of the outer catheter body 31. The outer hub 32 may be composed of a hollow cylinder which is circular or elliptic in cross-section. In addition, a wall part (tube wall) of the outer hub 32 may be equipped with an insertion port into which a finger or the like is inserted at the time of gripping the inner catheter 4 and operating it to move.

The inner catheter 4 is a catheter permitting insertion of the falloposcope 20 therethrough, and includes an inner catheter body 41 and an inner hub 42 fixed to a proximal portion of the inner catheter body 41.

The inner catheter body 41 is formed therein with a lumen 411 extending along the longitudinal direction (axial extent) of the inner catheter body 41. The falloposcope body 201 of the falloposcope 20 is inserted through the lumen 411.

The inner hub 42 is fixed to the proximal portion of the inner catheter body 41. The fixation method for effecting this fixation can be, for example, one of the fixation methods mentioned above for fixing the outer catheter body 31 and the outer hub 32. The inner catheter body 41 and the inner hub 42 may be integrally molded by, for example, injection molding or the like. The inner hub 42 is composed of a rectangular parallelepiped which is tetragonal in cross-section, and an inner cavity of the inner hub 42 communicates with the lumen 411 of the inner catheter body 41. The inner hub 42 may be composed of a hollow cylinder which is circular or elliptic in cross-section. The falloposcope 20 passes through the inner cavity of the inner hub 42 to reach the lumen 411 of the inner catheter body 41. In the configuration shown, the inner hub 42 is located in the inner cavity 321 of the outer hub 32, independently of the position of the inner catheter 4 relative to the outer catheter 3.

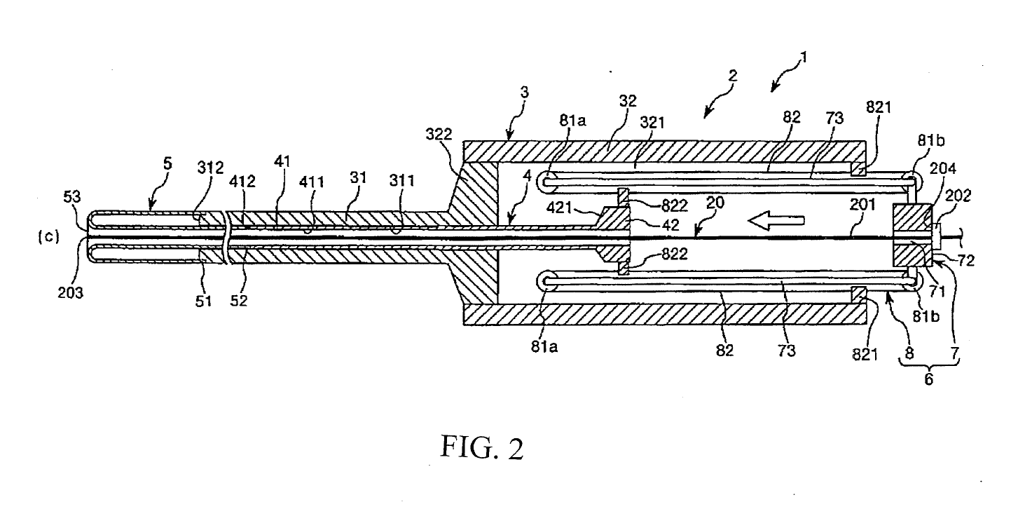

The inner catheter 4 thus configured can be axially moved relative to the outer catheter 3 in the longitudinal direction as seen from a comparison of FIGS. 1 and 2. In the balloon catheter assembly 1, when the inner catheter 4 is axially moved relative to the outer catheter 3, either the inner catheter 4 alone or both the inner catheter 4 and the falloposcope 20 together can be moved by an operation.

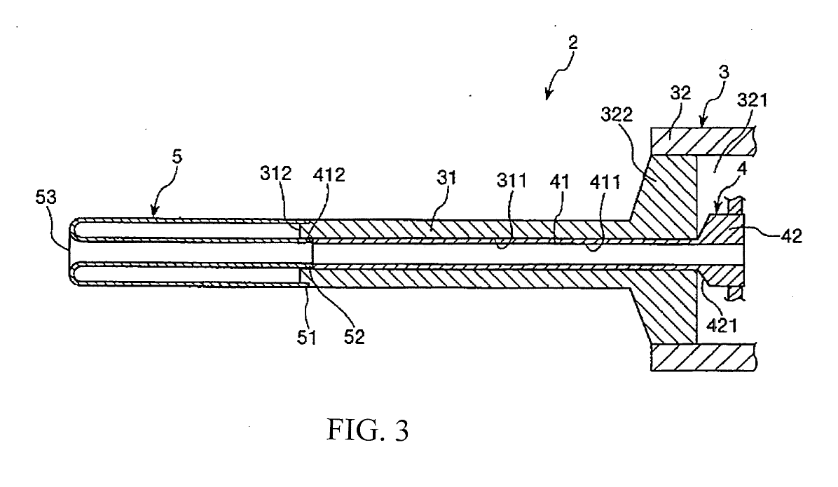

In addition, the inner hub 42 of the inner catheter 4 has an outside portion (outer diameter) greater than the inside diameter of the lumen 311 in the outer catheter body 31 of the outer catheter 3. This ensures that when the inner catheter 4 is axially moved relative to the outer catheter 3 in the distal direction, the distal end 421 of the inner hub 42 abuts on the distal end wall 322 of the outer hub 32 of the outer catheter 3 as seen in FIG. 3. This restricts or limits the movement of the inner catheter 4 in the distal direction, so that the distal end 412 of the inner catheter body 41 of the inner catheter 4 is prevented from projecting distally beyond the distal end 312 of the outer catheter body 31 of the outer catheter 3. Thus, the inner hub 42 also serves as a projection preventive means for preventing the distal end 412 of the inner catheter 4 from projecting distally beyond the distal end 312 of the outer catheter 3.

The material or materials constituting the outer catheter body 31 and the inner catheter body 41 are not specifically restricted, and, for example, various thermosetting or thermoplastic resins such as polyolefin resins, polyamide resins, urethane resins, polyimide resins, etc. can be used. Specific examples of the usable material(s) include polyolefins such as polyethylene, polypropylene, ethylene-propylene copolymer, ethylene-vinyl acetate copolymer (EVA), etc., polyvinyl chloride, polyesters such as polyethylene terephthalate (PET), polybutylene terephthalate (PBT), etc., polyurethane, polyamides, polyimides, polystyrene resins, fluoro-resins, and various thermoplastic elastomers based on styrene (polystyrene), polyolefin, polyvinyl chloride, polyurethane, polyester, polyamide, polybutadiene, fluoro-rubber or the like. Each of the outer catheter body 31 and the inner catheter body 41 may have a multilayer laminate structure composed of a plurality of materials.

The material or materials constituting the outer hub 32 and the inner hub 42 are not specifically restricted. Examples of the material(s) which can be used here include resin materials such as polyvinyl chloride, polyethylene, polypropylene, polycarbonate, polymethyl methacrylate, acrylonitrile-styrene-butadiene copolymer, etc. and various metallic materials.

The balloon 5 is constructed from a flexible film member. As shown in FIGS. 1 to 3, the balloon 5 is folded back at an intermediate portion thereof, has one end 51 fixed to the distal end 312 (distal portion) of the outer catheter 3, and has the other end 52 fixed to the distal end 412 (distal portion) of the inner catheter 4. This results in the balloon 5 having a bag-shaped form or configuration. The balloon 5 inflates upon supply of a working fluid and contracts upon removal of the working fluid by suction. The fixation method for fixing the balloon 5 to the distal end 312 (distal portion) of the outer catheter 3 and to the distal end 412 (distal portion) of the inner catheter 4 can be one of the fixation methods discussed above for fixing the outer catheter body 31 and the outer hub 32.

In addition, the balloon 5 is ring-shaped in cross-section so that the falloposcope 20 can be inserted through a central portion of the balloon 5.

As shown in FIGS. 1(a) and 1(b), when the inner catheter 4 is moved relative to the outer catheter 3, the position of the folded-back portion (distal end 53) of the balloon 5 varies along the longitudinal direction of the catheter according to a movement amount L1 of the inner catheter 4, so that a projection amount L2 of the balloon 5 beyond the distal end 312 of the outer catheter 3 also varies.

The balloon 5 is formed from one of various polymeric materials, particularly preferably a thermoplastic resin. In this case, preferably, the balloon 5 is formed from a material which is flexible as a whole but has a comparatively low percent elongation. Examples of the material which can be used to constitute the balloon 5 include: polyester resins or polyester elastomers such as polyethylene terephthalate, polybutylene terephthalate, etc.; olefin resins such as polyethylene, polypropylene, etc. which may be or may not be cross-linked by irradiation with electron rays; vinyl chloride resins; polyamide resins or polyamide elastomers such as nylon 11, nylon 12, nylon 610, etc.; polyurethane resins, ethylene-vinyl acetate copolymer which may be or may not be cross-linked by irradiation with electron rays; and polymer blends, polymer alloys and the like containing at least one of these polymers.

The balloon catheter 2 further has position restriction means 6 for restricting the position of the distal end 203 of the falloposcope body 201 of the falloposcope 20, which is inserted through the inner catheter 4, relative to the distal-most end 53 of the balloon 5. Set forth below is a description of the position restriction means 6.

As shown in FIGS. 1(a), 1(b) and 2, the position restriction means 6 is composed of a contact member 7 disposed on the proximal side of the inner catheter, and a connection mechanism 8 by which the contact member 7 and the inner catheter 4 are connected to each other so that they can be brought close to and away from each other. In the illustrated embodiment, the connection mechanism is a connector that operatively connects the contact member 7 and the inner catheter 4 so that movement of the inner catheter 4 causes movement of the contact member 7.

The contact member 7 is composed of a member which can be block-shaped. In addition, the contact member 7 includes a through-hole 71 penetrating the contact member 7 in the thickness direction (in the longitudinal direction of the catheter). The through-hole 71 permits the falloposcope body 201 of the falloposcope 20 to be inserted therethrough. When inserting the falloposcope 20 through the balloon catheter 2, the falloposcope body 201 of the falloposcope 20 is first inserted through the through-hole 71 in the contact member 7 from the proximal side. Further, the falloposcope body 201 is inserted through the inner catheter 4. In this manner, the inserting operation is carried out.

The proximal end face of the contact member 7 constitutes a contact surface 72 which the enlarged-diameter section 202 of the falloposcope 20 can contact.

The material constituting the contact member 7 is not specifically restricted. Examples of materials which can be used to fabricate the contact member 7 include the same material discussed above for the outer hub 32 or the inner hub 42.

The connection mechanism 8 includes two pulley-wire sets, each of which includes a pair of pulleys 81a and 81b, and a linear member or wire 82 wrapped around the pulley 81a and the pulley 81b. The number of pulley-wire sets each including the pair of pulleys 81a, 81b and the wire 82 is not limited to two, and the number may be three or more, or may be only one. The example discussed in more detail below involves a structure in which two pulley-wire sets are arranged. In this balloon catheter 2, the two pulley-wire sets are disposed correspondingly on both sides of the center axis of the inner catheter 4 (on the upper side and the lower side in FIGS. 1 and 2). The two pulley-wire sets are the same in configuration, and, therefore, one of the two sets will be described below. The same description applies to the other set.

The pulley 81a and the pulley 81b are spaced from each other along the longitudinal direction of the catheter, and are rotatably supported on a pulley support section (arm) 73 of the contact member 7. In addition, the pulley 81a is disposed on the inside of the outer hub 32 of the outer catheter 3, whereas the pulley 81b is disposed on the outside of the outer hub 32.

The wire 82 is, for example, a bundle (strand) of a multiplicity of metallic wires. The wire 82 is equipped, at intermediate parts thereof, with an outer hub fixing part 821 for fixing the outer hub 32 of the outer catheter 3, and with an inner hub fixing part 822 for fixing the inner hub 42 of the inner catheter 4. The outer hub fixing part 821 is a part by which an inner peripheral portion of the proximal end of the outer hub 32 of the outer catheter 3 and the wire 82 are connected and fixed to each other, while the inner hub fixing part 822 is a part by which an outer peripheral portion of the proximal end of the inner hub 42 of the inner catheter 4 and the wire 82 are connected and fixed to each other. The member wrapped around the pulleys 81a and 81b is not limited to the wire 82, but may be a belt-shaped member such as a belt, for example.

With the position restriction means 6 configured as above, when the inner catheter 4 is gripped and is moved in the distal axial direction by the movement amount L1, the contact member 7 is also moved in the same direction attendantly on this movement as seen from a comparison of FIGS. 1(a) and 1(b). As above-mentioned, the inner catheter 4 and the contact member 7 are connected to each other by the connection mechanism 8 having the two sets of the pulleys 81a, 81b and the wire 82. Therefore, the movement amount L3 of the contact member 7 is equal to L1/2.

Because the balloon 5 is folded back at an intermediate portion thereof as above-mentioned, when the inner catheter 4 is moved in the distal axial direction by the movement amount L1, the projection amount L2 is equal to L1/2 as shown in FIGS. 1(a) and 1(b). Thus, when the inner catheter 4 is moved in the distal axial direction, the movement amount L3 of the contact member 7 and the projection amount L2 of the balloon 5 are equal to each other. This helps enable the operator to easily grasp or understand the projection amount L2 of the balloon 5.

In a first state (an initial state) shown in FIG. 1(a), the balloon catheter assembly 1 is in such a state that a distal end face 204 of the enlarged-diameter section 202 of the falloposcope 20 is in abutment on the contact surface 72 of the contact member 7 of the balloon catheter assembly 1 so that the distal-most end 53 of the balloon 5 and the distal end 203 of the falloposcope 20 are located at the same position with respect to the longitudinal direction of the catheter.

Then, when the inner catheter 4 is moved by the movement amount L1 in the distal direction starting from the state shown in FIG. 1(a), a second state shown in FIG. 1(b) is established. In this second state, the contact member 7 has been moved by the movement amount L3, which is one half the movement amount L1. And the balloon 5 project by the projection amount L2, which is one half the movement amount L1.

Furthermore, as shown in FIG. 2, starting from the state shown in FIG. 1(b), the falloposcope 20 is moved in the distal direction until the enlarged-diameter section 202 abuts the contact member 7. The amount of movement of the falloposcope 20 in this case is L1/2, naturally. In the third state shown in FIG. 2, therefore, the distal end 203 of the falloposcope 20 and the distal-most end 53 of the balloon 5 are located at the same position relative to one another with respect to the longitudinal direction of the catheter as in the state shown in FIG. 1(a).

The balloon catheter is thus constructed so that the inner catheter 4 can axially move in the distal direction relative to the outer catheter 3 from a first position shown in FIG. 1(a) to a second position shown in FIG. 1(b) spaced apart from each other by a first axial distance L1 and this movement causes the balloon 5 to project further distally beyond the distal-most end 312 of the outer catheter 3. An axial distance between the distal-most end 53 of the balloon 5 at the first position shown in FIG. 1(a) and the distal-most end 53 of the balloon 5 at the second position shown in FIG. 1(b) is a second distance L2 that is less than the first distance L1. In addition, the connection mechanism 8 (connector) connects the contact member 7 (contact portion) and the inner catheter 4 so that the axial movement of the inner catheter 4 relative to the outer catheter 3 over the first axial distance L1 causes movement of the contact portion 7 over an axial distance no greater than the second axial distance L2.

Thus, the balloon catheter assembly 1 is constructed in such a way that at the limit of movement of the falloposcope 20 in the distal direction of the falloposcope 20, the distal end 203 of the falloposcope 20 is located at the distal-most end 53 of the balloon 5. Consequently, the distal end 203 of the falloposcope 20, which is inserted through the inner catheter 4 of the balloon catheter assembly 1, is securely prevented from projecting in excess from the distal-most end 53 of the balloon 5 (i.e., is prevented from projecting distally beyond the distal-most end 53 of the balloon 5).

If, by way of example, the distal end 203 of the falloposcope 20 projected in excess from the distal-most end 53 of the balloon 5, the distal end 203 of the falloposcope 20 having thus projected may impinge on the fallopian tube to damage the fallopian tube. Or, on the contrary, the distal end 203 of the falloposcope 20 having impinged on the fallopian tube may be damaged (cracked).

The balloon catheter assembly 1 disclosed here can reliably inhibit or prevent this from occurring because the distal end 203 of the falloposcope 20 is prevented from projecting from the distal-most end 53 of the balloon 5.

In addition, as above-mentioned, the distal end 412 of the inner catheter 4 is also prevented from projecting beyond the distal end 312 of the outer catheter 3. This helps ensure that in the balloon catheter assembly 1, none of the components, inclusive of the falloposcope 20, projects beyond the balloon 5. Therefore, such troubles as mentioned above can be more reliably prevented from occurring.

Now, an example of the method of using the balloon catheter assembly 1 will be described in detail below.

First, as shown in FIG. 10(a), the balloon catheter assembly 1 is set in the first state previously shown and described in FIG. 1(a). Keeping this state, the balloon catheter assembly 1 is inserted into a fallopian tube 30 to come into the vicinity of a stenosed part 301 of the fallopian tube 30. This inserting operation can be performed under X-ray fluoroscopy, for example. In addition, during the inserting operation, the distal-most end 203 of the falloposcope 20 is prevented from projecting distally beyond the distal-most end 53 of the balloon 5, as discussed above. Therefore, damage to the fallopian tube 30 by the distal end 203 is prevented from occurring.

Next, as shown in FIG. 10(b), the inner catheter 4 is moved forward (advanced), to establish the second state previously shown and described in FIG. 1(b). The balloon 5 is thus shifted or moved so that it projects distally farther beyond the distal end 312 of the outer catheter body 31 than in the FIG. 10(a) state. Concurrently with this projecting operation, a working fluid is supplied into the balloon 5. This reliably or assuredly sets the balloon 5 in the projecting state. Consequently, the stenosed part 301 can be enlarged by the balloon 5 in this projecting state.

Subsequently, as shown in FIG. 10(c), the inner catheter 4 is moved backward (withdrawn), resulting in that the stenosed part 301 which has been enlarged can be observed through the distal end 203 of the falloposcope 20 located at the distal end 53 of the balloon 5.

In a situation in which it is desired to insert the balloon catheter assembly 1 further into (deeper into) to the fallopian tube 30 starting from the state shown in FIG. 10(b) without performing observation of the stenosed part 301 which has been enlarged, the falloposcope 20 is moved forward (advanced) into the third state previously shown and described in FIG. 2, whereby the inserting operation can be carried out. Then, in the third state, the inside of the fallopian tube 30 can be observed through the distal end 203 of the falloposcope 20 located at the distal end 53 of the balloon 5. Also in the third state, the distal end 203 of the falloposcope 20 is inhibited or prevented from projecting beyond the distal end 53 of the balloon 5 and so the possibility of damaging the fallopian tube 30 by the distal end 203 is prevented from occurring.

FIGS. 4(a) and 4(b) illustrate a second embodiment of the balloon catheter assembly (balloon catheter) disclosed here. The following description primarily focuses on differences between this embodiment and the earlier described embodiment. Features in this second embodiment which are similar to the first embodiment are identified by a common reference numeral, and a detailed description of such features is not repeated.

This second embodiment is the same as the first embodiment above, except for the configuration of the connection mechanism in the position restriction means.

As shown in FIG. 4, the position restriction means 6A in this embodiment includes a connection mechanism 8A having two pinion gear-rack sets, each including a pinion gear 83, an inside rack 84 provided on the inner catheter 4 (provided on the outer surface of the inner hub 42), and an outside rack 85 provided on the outer catheter 3 (provided on the inner surface of the outer hub 32). The number of the pinion gear-rack sets each including the pinion gear 83, the inside rack 84 and the outside rack 85 is not limited to two, as the number may be three or more, or may be only one. This embodiment disclosed by way of example here and discussed in more detail below includes a structure in which two pinion gear-rack sets are included. In this balloon catheter 2, the pinion gear-rack sets are arranged correspondingly on both sides of the center axis of the inner catheter 4 (the upper side and the lower side in FIG. 4). The two sets are the same as one another and so one of the two sets will be described below, it being understood that the description applies equally to the other set.

The pinion gear 83 is rotatably supported on a distal-side portion of the contact member 7, through a pinion gear support section (arm) 74.

The inside rack 84 is provided at the outer peripheral portion or surface of the inner hub 42 of the inner catheter 4. The inside rack 84 engages the pinion gear 83.

The outside rack 85 is provided at the inner peripheral portion or surface of the outer hub 32 of the outer catheter 3. In addition, the outside rack 85 faces the inside rack 84, with the pinion gear 83 therebetween. The outside rack 85 engages the pinion gear 83, like the inside rack 84.

Also in the position restriction means 6A thus configured, like in the position restriction means 6 in the first embodiment above, movement of the inner catheter 4 in the distal direction by the movement amount L1 results in that the movement amount L3 of the contact member 7 is equal to L1/2 (see FIG. 4). The projection amount L2 of the balloon 5 is equal to L1/2 (FIG. 4). Thus, when the inner catheter 4 is moved in the distal direction, the movement amount L3 of the contact member 7 and the projection amount L2 of the balloon 5 are equal to each other. This helps enable the operator to relatively easily grasp or understand the projection amount L2 of the balloon 5.

In a first state shown in FIG. 4(a), the balloon catheter assembly 1 is in such a state that the distal end face 204 of the enlarged-diameter section 202 of the falloposcope 20 is in contact with the contact surface 72 of the contact member 7 of the balloon catheter assembly 1 so that the distal end 53 of the balloon 5 and the distal end 203 of the falloposcope 20 are located at the same position with respect to the longitudinal or axial direction of the catheter.

Then, in a second state shown in FIG. 4(b) established when the inner catheter 4 is moved in the distal axial direction by the movement amount L1 starting from the state shown in FIG. 4(a), the contact member 7 moves by the movement amount L3, which is equal to one half the movement amount L1. The balloon 5 projects by the projection amount L2, which is equal to one half the movement amount L1.

Further, when the falloposcope 20 is moved in the distal direction until the enlarged-diameter section 202 abuts the contact member 7 starting from the state shown in FIG. 4(b), the distal end 203 of the falloposcope 20 and the distal end 53 of the balloon 5 are once again located at the same position with respect to the longitudinal direction of the catheter.

Thus, also in the balloon catheter assembly 1 in this embodiment, it is possible to achieve a restriction such that at the limit of movement of the falloposcope 20 in the distal direction, the distal end 203 of the falloposcope 20 is located at the distal end 53 of the balloon 5. This ensures that the distal end 203 of the falloposcope 20, which is inserted through the inner catheter 4 of the balloon catheter assembly 1, is securely prevented from projecting distally beyond the distal-most end 53 of the balloon 5.

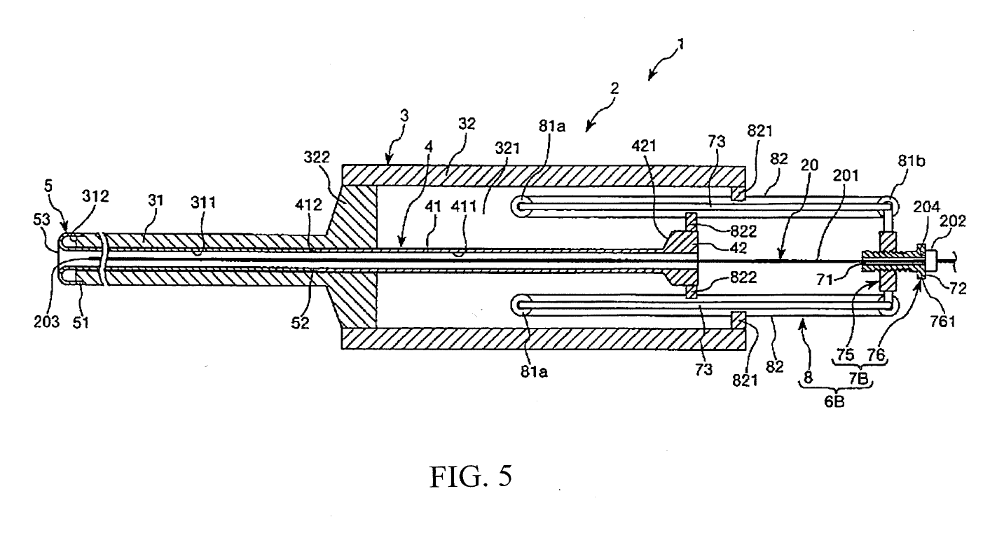

FIG. 5 illustrates a third embodiment of the balloon catheter assembly (balloon catheter) disclosed here. The following description primarily focuses on differences between this embodiment and the earlier described embodiments. Features in this third embodiment which are similar to features in the earlier embodiments are identified by a common reference numeral, and a detailed description of such features is not repeated.

This third embodiment is the same as the first embodiment above, except for the configuration of the contact member of the position restriction means.

As shown in FIG. 5, the position restriction means 6B includes a contact member 7B composed of a body section 75 and a bolt 76 which is in screw engagement (threaded engagement) with the body section 75 which is provided with pulley support sections 73.

A through-hole 71 is provided in the bolt 76 and penetrates the bolt 76 in the longitudinal direction of the bolt. The through-hole 71 permits the falloposcope body 201 of the falloposcope 20 to be inserted therethrough. In addition, a top face (proximal end face) of a head section (screw head) 761 of the bolt 76 constitutes the contact surface 72 capable of making contact with the enlarged-diameter section 202 of the falloposcope 20. The bolt 76 constitutes an adjustment mechanism permitting adjustment of the position of the contact surface in the longitudinal direction of the catheter.

In the contact member 7B thus configured, when the bolt 76 is rotated in a predetermined direction relative to the body section 75, the position of the contact surface 72 can be adjusted or moved toward the proximal side. When the bolt 76 is rotated in the reverse or opposite direction to the above-mentioned direction, the position of the contact surface 72 can be adjusted or moved toward the distal side. In this manner, the position of the contact surface 72 can be finely adjusted along the longitudinal direction of the catheter. This helps ensure that the position of the distal end 203 of the falloposcope 20 relative to the distal end 53 of the balloon 5 in the first state can be adjusted or varied, for example so that the distal-most end 203 of the falloposcope 20 is located a little on the proximal side relative to the distal-most end 53 of the balloon 5 such as shown in FIG. 5. In addition, the position can also be adjusted so that the distal-most end 203 of the falloposcope 20 is located a little on the distal side relative to the distal-most end 53 of the balloon 5.

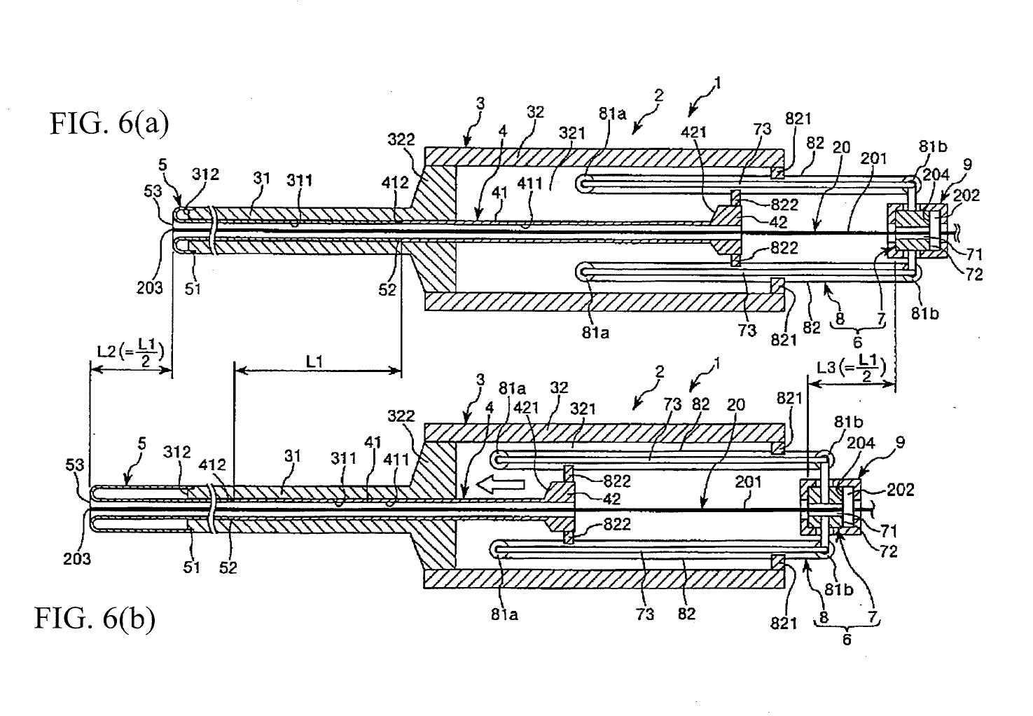

FIG. 6 illustrates a fourth embodiment of the balloon catheter assembly (balloon catheter) disclosed here. The following description primarily focuses on differences between this embodiment and the earlier described embodiments. Features in this fourth embodiment which are similar to features in the above-described embodiments are identified by common reference numerals, and a detailed description of such features is not repeated.

This fourth embodiment is the same as the first embodiment above, except that the position restriction means of the balloon catheter further includes a lock member.

As shown in FIG. 6, the position restriction means of the balloon catheter 2 further has a lock member 9 for maintaining a state in which the contact member 7 and the enlarged-diameter section 202 of the falloposcope 20 are in contact with each other.

The lock member 9 clamps the contact member 7 and the enlarged-diameter section 202 of the falloposcope 20 from the distal side and the proximal side. This helps makes it possible to securely maintain the state in which the contact member 7 and the enlarged-diameter section 202 of the falloposcope 20 are in contact with each other. Accordingly, it is possible to reliably maintain a state in which the distal end 53 of the balloon 5 and the distal end 203 of the falloposcope 20 are located at the same position with respect to the longitudinal direction of the catheter.

Starting from the state shown in FIG. 6(a) and performing an operation of moving the lock member 9 in the distal direction, the contact member 7 and the falloposcope 20 move together as seen from a comparison of FIGS. 6(a) and 6(b). Therefore, operationality of the balloon catheter 2 is enhanced, as compared with the case where the contact member 7 and the falloposcope 20 are operated separately. In addition, during the operation of moving the lock member 9, the state in which the distal end 53 of the balloon 5 and the distal end 203 of the falloposcope 20 are located at the same position with respect to the longitudinal or axial direction of the catheter is constantly maintained. Consequently, the balloon 5 can be moved while observing the inside of the fallopian tube 30 through the falloposcope 20.

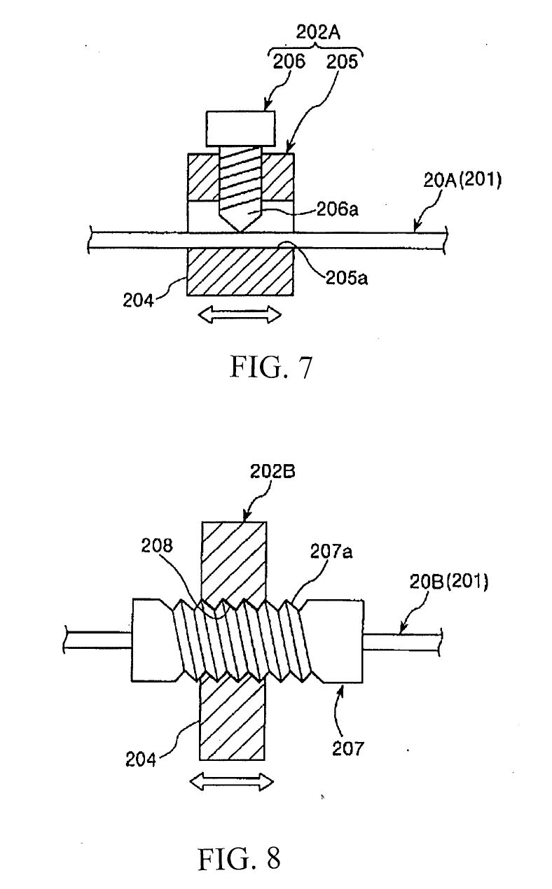

FIG. 7 illustrates a fifth embodiment of the balloon catheter assembly (balloon catheter) disclosed here. The following description primarily focuses on differences between this embodiment and the earlier described embodiments. Features in this fifth embodiment which are similar to features in the above-described embodiments are identified by common reference numerals, and a detailed description of such features is not repeated.

This fifth embodiment is the same as the first embodiment above, except for a difference in the configuration of the enlarged-diameter section of the falloposcope.

As shown in FIG. 7, the falloposcope 20A includes an enlarged-diameter section or enlarged member 202A which is composed of a body separate from the falloposcope body 201. The enlarged-diameter section 202A is composed of a body section 205 and a bolt 206 in screw engagement with the body section 205.

The body section 205 is annular-shaped or ring-shaped. The inside diameter of the body section 205 is greater than the outside diameter of the falloposcope body 201.

The bolt 206 is in screw engagement with a wall part of the body section 205. In addition, a leg section (screw section) 206a of the bolt 206 can contact and clamp the falloposcope body 201 between the inner peripheral portion 205a of the body section 205 and the leg section 206a as shown in FIG. 7.

When the bolt 206 is rotated in a predetermined direction relative to the body section 205, the bolt 206 is loosened so that the leg section 206a moves away from the falloposcope body 201. This permits the enlarged-diameter section 202A as a whole to move along the longitudinal direction of the falloposcope body 201, so that the position of the enlarged-diameter section 202A can be finely adjusted. Then, with the bolt 206 in the finely adjusted position and rotated in the reverse or opposite direction to the above-mentioned direction, movement of the enlarged-diameter section 202A is restricted. This helps ensure that the position of the distal end 203 of the falloposcope 20A relative to the distal end 53 of the balloon 5 in the first state can be adjusted so that, for example, the distal-most end 203 of the falloposcope 20A is located a little on the proximal side relative to the distal-most end 53 of the balloon 5. In addition, the position can also be adjusted so that the distal-most end 203 of the falloposcope 20A is located a little on the distal side relative to the distal-most end 53 of the balloon 5. Consequently, the position of the distal end 203 of the falloposcope 20A relative to the distal end 53 of the balloon 5 can be finely adjusted according to the operator's preference or the nature of the intended procedure.

FIG. 8 illustrates a sixth embodiment of the balloon catheter assembly (balloon catheter) disclosed here. The following description primarily focuses on differences between this embodiment and the earlier described embodiments. Features in this sixth embodiment which are similar to features in the above-described embodiments are identified by common reference numerals, and a detailed description of such features is not repeated.

This sixth embodiment is the same as the fifth embodiment above, except for a difference in the configuration of the enlarged-diameter section of the falloposcope.

As shown in FIG. 8, the falloposcope 20B includes an enlarged-diameter section or enlarged member 202B connected to the falloposcope body 201 through a connection member 207.

The connection member 207, is cylindrical in outer shape and is fixed to the falloposcope body 201. The outer peripheral portion of the connection member 207 includes a male screw 207a.

The enlarged-diameter section 202B is annular-shaped or ring-shaped. The inner peripheral surface of the through hole in the enlarged-diameter section 202B is screw-threaded to form a female screw 208 for threaded engagement with the male screw 207a of the connection member 207.

In the falloposcope 20B thus configured, when the enlarged-diameter section 202B is rotated in a predetermined direction relative to the connection member 207, the enlarged-diameter section 202B is moved toward the proximal side or in the proximal direction. In addition, when the enlarged-diameter section 202B is rotated in the reverse or opposite direction to the above-mentioned direction, the enlarged-diameter section 202B moves toward the distal side. Thus, the position of the enlarged-diameter section 202B can be finely adjusted in the longitudinal or axial direction of the catheter. This helps ensure that the position of the distal end 203 of the falloposcope 20B relative to the distal-most end 53 of the balloon 5 in the first state can be adjusted so that, for example the distal end 203 of the falloposcope 20B is located a little on the proximal side relative to the distal-most end 53 of the balloon 5. The position can also be adjusted so that the distal-most end 203 of the falloposcope 20B is located a little on the distal side relative to the distal-most end 53 of the balloon 5. Consequently, the position of the distal-most end 203 of the falloposcope 20B relative to the distal-most end 53 of the balloon 5 can be finely adjusted according to the operator's preference or the nature of the intended procedure.

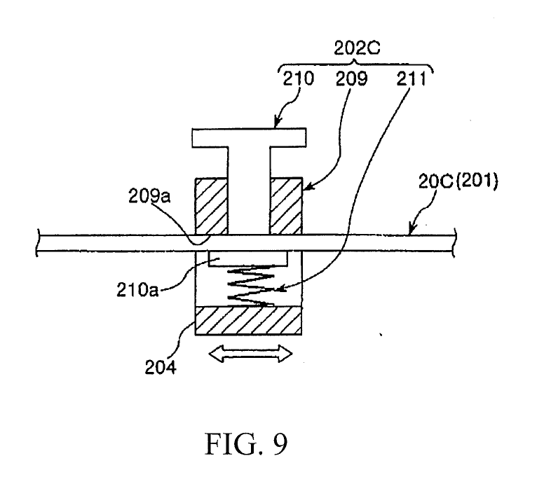

FIG. 9 illustrates a seventh embodiment of the balloon catheter assembly (balloon catheter) disclosed here. The following description primarily focuses on differences between this embodiment and the earlier described embodiments. Features in this seventh embodiment which are similar to features in the above-described embodiments are identified by common reference numerals, and a detailed description of such features is not repeated.

This embodiment is the same as the fifth embodiment above, except for a difference in the configuration of the enlarged-diameter section of the falloposcope.

As shown in FIG. 9, the falloposcope 20C includes an enlarged-diameter section or enlarged member 202C composed of a body section 209, a pin 210 fitted in a through hole in the body section 209, and a coil spring 211 also positioned in the through hole of the body section and biasing the pin 210.

The body section 209 is ring-shaped or annular-shaped. The inside diameter of the through hole in the body section 209 is greater than the outside diameter of the falloposcope body 201.

The pin 210 is fitted in the wall part of the body section 209 in such a manner as to be movable in a direction orthogonal to the axial direction of the body section 209. In addition, a flange section 210a of the pin 210 can clamp the falloposcope body 201 between an inner peripheral portion 209a of the through hole in the body section 209 and the flange section 210a of the pin 210 as shown in FIG. 9.

The coil spring 211 biases the pin 210 in a direction such that the flange section 210a clamps the falloposcope body 201 between the inner peripheral portion 209a of the body section 209 and the flange section 210a of the pin 210. This helps ensure that the falloposcope body 201 is clamped assuredly. Movement of the enlarged-diameter section 202C along the longitudinal or axial direction of the falloposcope body 201 is thus restricted.

In the falloposcope 20C thus configured, when the pin 210 is depressed against the biasing force of the coil spring 211, the clamping force exerted on the falloposcope body 201 is released. As a result, the enlarged-diameter section 202C as a whole can be moved along the longitudinal direction of the falloposcope body 201, so that the position of the enlarged-diameter section 202C can be finely adjusted. Then, when the depressing force on the pin 210 in the finely adjusted position is released, movement of the enlarged-diameter section 202C is restricted as above-mentioned. This helps ensure that the position of the distal end 203 of the falloposcope 20C relative to the distal end 53 of the balloon 5 in the first state can be adjusted so that, for example, the distal end 203 of the falloposcope 20C is located a little on the proximal side relative to the distal end 53 of the balloon 5. In addition, the position can also be adjusted so that, on the contrary to the above, the distal end 203 of the falloposcope 20C is located a little on the distal side relative to the distal end 53 of the balloon 5. Consequently, the position of the distal end 203 of the falloposcope 20C relative to the distal end 53 of the balloon 5 can be finely adjusted according to the operator's preference or the nature of the intended procedure.

While the balloon catheter and the balloon catheter assembly disclosed here have been described above based on embodiments shown in the drawings, the invention is not limited to the above embodiments. Components of the balloon catheter and the balloon catheter assembly can be replaced by alternatives that can exhibit the same or similar functions. And other features may be added.

In addition, the balloon catheter and the balloon catheter assembly may combine arbitrary two or more configurations (characteristic features) of the above-described embodiments. For example, the embodiments shown in FIGS. 1-4 can be used with the adjustment mechanisms shown in FIGS. 5 and 7-9, and the embodiment shown in FIG. 6 can be used with the connection mechanism 8A shown in FIG. 4.

The balloon catheter assembly disclosed here may be an assembly in which the contact member disposed on the proximal side of the inner catheter and a medical elongate member are inseparably integrated with each other.

In addition, the balloon catheter may be provided with graduations for indicating the amount of movement when the inner catheter is operated to move. Or, alternatively, a graduation member having the function as graduations may be provided. The graduation member is not specifically restricted; for example, a bellows-like member can be preferably used.

While the falloposcope has been mentioned in the above-described embodiments as an example of the medical elongate member to be inserted through the balloon catheter, this is not limitative. Other examples of the medical elongate member include other kinds of endoscopes, catheters, and guide wires.

The balloon catheter disclosed here includes: an outer catheter; an inner catheter inserted in the outer catheter, capable of moving relative to the outer catheter in the longitudinal direction thereof, and permitting a flexible medical elongate member to be inserted therethrough; an inflatable and contractible balloon constructed from a film member, having one end portion and the other end portion fixed respectively to a distal portion of the outer catheter and a distal portion of the inner catheter, and projecting from the distal end of the outer catheter by an amount which varies according to the amount of movement of the inner catheter relative to the outer catheter; and position restriction means for restricting the position of the distal end of the medical elongate member, which is inserted through the inner catheter, relative to the distal end of the balloon. Therefore, the medical elongate member inserted through the inner catheter of the balloon catheter can be prevented from projecting beyond or excessively beyond the distal end of the balloon. Accordingly, the balloon catheter disclosed here has industrial applicability.

The detailed description above describes features and aspects of embodiments, disclosed by way of example, of a balloon catheter and a balloon catheter assembly. The invention is not limited, however, to the precise embodiments and variations described and illustrated. Various changes, modifications and equivalents could be effected by one skilled in the art without departing from the spirit and scope of the invention as defined in the appended claims. It is expressly intended that all such changes, modifications and equivalents which fall within the scope of the claims are embraced by the claims.

What is claimed is:

1. A balloon catheter assembly comprising:
a longitudinally extending outer catheter possessing a distal portion;
an inner catheter positioned in the outer catheter and movable relative to the outer catheter in a longitudinal direction of the outer catheter, the inner catheter possessing a distal portion;
an inflatable and contractible balloon constructed from a film member, the film member having one end portion fixed to the distal portion of the outer catheter and an other end portion fixed to a distal portion of the inner catheter, the balloon projecting distally beyond a distal-most end of the outer catheter by an amount which varies according to an amount of relative longitudinal movement between the inner catheter and the outer catheter;
an elongated medical member positioned in the inner catheter of the balloon catheter and being movable relative to the inner catheter, the elongated medical member possessing a distal-most end; and
position restriction means for restricting a position of the distal-most end of the medical elongate member positioned in the inner catheter relative to the distal end of the balloon.

2. The balloon catheter assembly according to claim 1, wherein the position restriction means comprises a contact member having a through hole through which passes the elongated medical member.

3. The balloon catheter assembly according to claim 2, the position restriction means further comprising an or enlarged member on the elongated medical member, the enlarged member being movable together with the elongated medical member so that the enlarged member contacts the contact member during movement of the elongated medical member in a distal direction relative to the contact member.

4. The balloon catheter assembly according to claim 3, wherein the enlarged member is configured so that a position of the enlarged member on the elongated medical member is adjustable in a lengthwise direction of the elongated medical member.

5. The balloon catheter assembly according to claim 3, wherein the enlarged member is fixed in position on the elongated medical member, and the position restriction means further comprising a lock member which fixes the enlarged member in position relative to the contact member so that the enlarged member does not move relative to the contact member when the inner catheter is moved relative to the outer catheter in the longitudinal direction of the outer catheter.

6. The balloon catheter assembly according to claim 1, wherein the elongated medical member is a falloposcope.

7. A balloon catheter comprising:
a longitudinally extending outer catheter possessing a distal portion;
an inner catheter positioned in the outer catheter and axially movable relative to the outer catheter in a distal direction and proximal direction relative to the outer catheter, the inner catheter having a through hole configured to receive an elongated medical member, the inner catheter possessing a distal portion;
an inflatable and contractible balloon comprised of a film member and projecting distally beyond a distal-most end of the outer catheter, the film possessing one end portion fixed to the distal portion of the outer catheter and an other end portion fixed to the distal portion of the inner catheter so that axial movement of the inner catheter in the distal direction relative to the outer catheter from a first position to a second position spaced apart from each other by a first axial distance causes the balloon to project further distally beyond the distal-most end of the outer catheter, an axial distance between a distal-most end of the balloon at the first position and the distal-most end of the balloon at the second position being a second distance less than the first distance;
a contact portion configured to be contacted by a part of the elongated medical member when the elongated medical member is moved relative to the contact portion; and
a connector connecting the contact portion and the inner catheter so that the axial movement of the inner catheter relative to the outer catheter over the first axial distance causes movement of the contact portion over an axial distance no greater than the second axial distance.

8. The balloon catheter according to claim 7, wherein the connector is configured such that the axial movement of the inner catheter relative to the outer catheter over the first axial distance causes the contact portion to axially move one-half the first axial distance.

9. The balloon catheter according to claim 7, wherein the connector comprises: at least one pair of pulleys connected to the contact member and extending in a longitudinal direction of the inner catheter; and a linear member extending around the pair of pulleys, the inner catheter and the outer catheter both being fixed to the linear member.

10. The balloon catheter according to claim 7, wherein the connector comprises: at least one gear rotatably supported on an arm connected to the contact member; an inside rack provided on the inner catheter; and an outside rack provided on the outer catheter and facing the inside rack, the gear being positioned between the inside rack and the outside rack, the gear being in contact with both the inside rack and the outside rack.

11. A balloon catheter comprising:
a longitudinally extending outer catheter;
an inner catheter positioned in the outer catheter, movable relative to the outer catheter in a longitudinal direction of the outer catheter, and configured to permit a flexible medical elongate member to be inserted therethrough;
an inflatable and contractible balloon constructed from a film member, the film member having one end portion fixed to a distal portion of the outer catheter and an other end portion fixed to a distal portion of the inner catheter, the balloon projecting from a distal end of the outer catheter by an amount which varies according to an amount of movement of the inner catheter relative to the outer catheter; and
position restriction means for restricting a position of a distal-most end of the medical elongate member, which is inserted through the inner catheter, relative to the distal end of the balloon.

12. The balloon catheter according to claim 11, wherein the position restriction means restricts the position of the distal-most end of the medical elongate member so that, at a limit of movement of the medical elongate member in a distal direction, the distal-most end of the medical elongate member is located in a vicinity of the distal end of the balloon.

13. The balloon catheter according to claim 11, wherein the position restriction means restricts the position of the distal-most end of the medical elongate member so that, at a limit of movement of the medical elongate member in a distal direction, the distal-most end of the medical elongate member is not located distally beyond a distal-most end of the balloon.

14. The balloon catheter according to claim 11, wherein the position restriction means comprises:
a contact member disposed on a proximal side of the inner catheter, the contact member possessing a contact surface contactable by a part of the medical elongate member; and
a connector by which the contact member and the inner catheter are connected to each other in a manner allowing the contact member and the inner catheter to be moved toward and away from each other.

15. The balloon catheter according to claim 14, wherein the connector is configured such that when the inner catheter and the contact member are moved in the same direction, an amount of movement of the contact member is one-half the amount of movement of the inner catheter.

16. The balloon catheter according to claim 14, wherein the connector comprises:
at least a pair of pulleys arranged on the contact member and extending in a longitudinal direction of the inner catheter; and
a linear member extending around the pair of pulleys, the inner catheter and the outer catheter both being fixed to the linear member.

17. The balloon catheter according to claim 14, wherein the connector comprises:
at least one pinion gear rotatably supported on the contact member;
an inside rack provided on the inner catheter; and
an outside rack provided on the outer catheter and facing the inside rack, the pinion being positioned between the inside rack and the outside rack, the pinion being in contact with both the inside rack and the outside rack.

18. The balloon catheter according to claim 14, wherein the contact member includes an adjustment mechanism allowing a position of the contact surface to be adjusted along a longitudinal direction of the contact member.

19. The balloon catheter according to claim 14, the position restriction means further comprising a lock member for maintaining a state in which the contact member and a part of the medical elongate member are in contact with each other when the inner catheter is moved distally in the longitudinal direction relative to the outer catheter.

20. The balloon catheter according to claim 11, comprising projection preventive means for preventing a distal-most end of the inner catheter from projecting distally beyond a distal end of the outer catheter.
